(19) United States
(12) Patent Application Publication
(10) Pub. No.: US 2018/0201916 A1
(43) Pub. Date: Jul. 19, 2018

(54) VARIANTS OF THE SUBTILISIN CARLSBERG POLYPEPTIDE WITH DECREASED THERMOSTABILITY

(71) Applicant: UVic Industry Partnerships Inc., Victoria (CA)

(72) Inventor: Francis Edward Nano, Victoria (CA)

(73) Assignee: UVic Industry Partnerships Inc., Victoria, BC (CA)

(21) Appl. No.: 15/742,145

(22) PCT Filed: Jul. 6, 2016
(86) PCT No.: PCT/IB2016/054055

§ 371 Date: Jan. 5, 2018

Related U.S. Application Data
(60) Provisional application No. 62/188,969, filed on Jul. 6, 2015.

(51) Int. Cl. C12N 9/54; C12Q 1/6806; C11D 3/386

(52) U.S. Cl. CPC C12N 9/54; C11D 3/386; C12Q 1/6806; C12Y 304/21062

(57) ABSTRACT

Heat labile Subtilisin Carlsberg polypeptide variants having SEQ ID No.s: 10, 14 and 18 are described. After protease digestion of a target polypeptide in a sample, these variants can conveniently be heat inactivated.

### FIELD OF INVENTION

The present invention relates to subtilisin variants and uses thereof. More specifically, the present invention relates to variant subtilisin Carlsberg sequences and uses thereof.

### BACKGROUND OF THE INVENTION

Enzymes that denature with moderate heat have an established role in molecular biology. Their time and cost-saving advantage comes from eliminating the need to do a purification step after the role of the enzyme is done. One simply uses a heat-inactivation step; the enzyme denatures, and further manipulations of the bio-molecules or cell-extract can occur. One example is the use of heat-labile restriction enzymes to digest DNA prior to ligation. Useful restriction enzymes include variants that digest DNA at 37° C., and can be heat denatured afterwards, typically by a twenty-minute heating step at 65° C. The alternatives to heat denaturation generally take longer, cost more and result in loss of DNA.

Commonly used proteases include proteinase K and subtilisin. Subtilisin-like proteases have been developed to be active in hot water for stain removers in laundry (U.S. Pat. No. 8,753,861 B2 entitled “Protease comprising one or more combinable mutations”; Kristjansson M M. 2012. Thermostable subtilases (subtilisin-like serine proteinases), p 67-105. In Sen S, Nilsson L (ed), Thermostable proteins: structural stability and design, 1st ed. CRC Press, Boca Raton, Fla.).

A naturally occurring heat-labile metalloprotease (a class of protease different from subtilisin proteases) from a cold ocean water bacterium called A9 was described by Moran et al. (Moran A. J et al. “Heat-labile proteases in molecular biology applications.” FEMS Microbiology Letters 187(1): 59-63, 2001). This enzyme is both cold-adapted (has good catalytic activity at cold temperature) and heat-labile (denatures at relatively low temperature) but does not have stability at working temperatures and is prone to autolysis, making its use limited. Directed evolution was used to mutate a similar psychrophilic enzyme, S41 (Davail, S. et al. “Cold Adaptation of Proteins” J. Biol. Chem., 269:17448-17453, 1994) to improve its thermostability and activity while retaining its activity at cool temperatures (Miyazaki, K. et al. “Directed Evolution Study of Temperature Adaptation in a Psychrophilic Enzyme,” J. Mol. Biol 297:1015-1026, 2000) and a mesophilic enzyme, subtilisin SSII, was also mutated to have low-temperature activity (Wintrode P. L., et al. “Cold Adaptation of a Mesophilic Subtilisin-like Protease by Laboratory Evolution.” J. Biol. Chem. 275: 31635-31640, 2000), in studies exploring the evolutionary process of cold or heat adaptation.

### SUMMARY OF THE INVENTION

The present invention relates to variant subtilisin Carlsberg sequences and uses thereof. In some aspects, the invention provides a variant, heat labile subtilisin Carlsberg polypeptide that includes a mutation at one or more of amino acids K88, D180, N181, N265, L321, L339 or Q379, or combinations thereof.

In some embodiments, the polypeptide includes a mutation at amino acids D180, L339 and Q379; L339 and Q379; D180 and L339; D180 and Q379; or K88, D180, N181, N265, and L321.

In some embodiments, the polypeptide includes the mutation at K88 is K88N, the mutation at D180 is D180G or D180A, the mutation at N181 is N181Y, the mutation at N265 is N265S, the mutation at L321 is L321 F, the mutation at L339 is L339M, or the mutation at Q379 is Q379P.

In some embodiments, the polypeptide includes the sequence set forth in any one of SEQ ID NOs: 9 to 14, 17 or 18.

In some aspects, the invention provides a nucleic acid molecule encoding a polypeptide as described herein.

In some aspects, the invention provides a nucleic acid molecule including the sequence set forth in in any one of SEQ ID NOs: 3 to 8, 15 or 16.

In some aspects, the invention provides an expression vector including a nucleic acid molecule as described herein.

In some aspects, the invention provides a host cell including a expression vector as described herein. The host cell may be a B. subtilis.

In some aspects, the invention provides a method of removing a target polypeptide from a sample by providing a sample including the target polypeptide and adding a polypeptide as described herein to the sample for a sufficient period of time and at a suitable temperature to remove the target polypeptide. The method may further include increasing the temperature of the sample (for example, to about 50° C.) to inactivate the polypeptide as described herein.

The target polypeptide may be a polypeptide used in molecular biology techniques, such as a heat resistant enzyme, a nuclease, a DNA modifying enzyme, a restriction enzyme, or a contaminant.

The sample may be a preparation of plasmid DNA, a preparation of chromosomal DNA, a preparation of mitochondrial DNA, a preparation of RNA, a forensic sample, a clinical sample, or a diagnostic sample.

In some aspects, the invention provides a composition comprising a polypeptide as described herein and a carrier. In some embodiments, the composition may be a detergent composition.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A shows the synthetic DNA encoding subtilisin Carlsberg used as a target for error prone PCR, with the start (ATG) and stop (TAA) codons underlined; spacer sequences shown in italics; the region encoding the added histidine tag shown in bold italics; codons corresponding to positions 88, 180, 181, 265, 321, 339 and 379 indicated in bold underline, and plasmid vector pZY167 sequences in lower case type (SEQ ID NO: 1);

FIG. 1B shows the polypeptide sequence of subtilisin Carlsberg, with the amino acid positions corresponding to amino acids 88, 180, 181, 265, 321, 339 and 379 indicated in bold (SEQ ID NO: 2);

FIG. 2A shows the DNA sequence of the ORF encoding subtilisin variant B24, with the start (ATG) and stop (TAA) codons underlined, the nucleotide changes indicated in bold, the associated codons underlined, and the region encoding the added histidine tag shown in bold italics (SEQ ID NO: 3);

FIG. 2B shows the cDNA sequence of the subtilisin variant B24, with the start (ATG) and stop (TAA) codons underlined, the nucleotide changes indicated in bold, and the associated codons underlined (SEQ ID NO: 4);

FIG. 2C shows the cDNA sequence of the subtilisin variant B24, with a G180D change and with the start (ATG) and stop (TAA) codons underlined, the nucleotide changes indicated in bold, the associated codons underlined, and the region encoding the added histidine tag shown in bold italics (SEQ ID NO: 5);

FIG. 2D shows the cDNA sequence of the subtilisin variant B24, with a G180D change and with the start (ATG) and stop (TAA) codons underlined, the nucleotide changes indicated in bold, and the associated codons underlined (SEQ ID NO: 6),

FIG. 2E shows the cDNA sequence of the subtilisin variant B24, with a G180A change and with the start (ATG) and stop (TAA) codons underlined, the nucleotide changes indicated in bold, the associated codons underlined, and the region encoding the added histidine tag shown in bold italics (SEQ ID NO: 7);

FIG. 2F shows the cDNA sequence of the subtilisin variant B24, with a G180A change and with the start (ATG) and stop (TAA) codons underlined, the nucleotide changes indicated in bold, and the associated codons underlined (SEQ ID NO: 8);

FIG. 2G shows the polypeptide sequence of the subtilisin variant B24, with the amino acid changes indicated in bold and the added histidine tag shown in bold italics (SEQ ID NO: 9);

FIG. 2H shows the polypeptide sequence of the subtilisin variant B24, with the amino acid changes indicated in bold (SEQ ID NO: 10);

FIG. 2I shows the polypeptide sequence of the subtilisin variant B24, with a G180D change and with the amino acid changes indicated in bold and the added histidine tag shown in bold italics (SEQ ID NO: 11);

FIG. 2J shows the polypeptide sequence of the subtilisin variant B24, with a G180D change and with the amino acid changes indicated in bold (SEQ ID NO: 12);

FIG. 2K shows the polypeptide sequence of the subtilisin variant B24, with a G180A change and with the amino acid changes indicated in bold and the added histidine tag shown in bold italics (SEQ ID NO: 13);

FIG. 2L shows the polypeptide sequence of the subtilisin variant B24, with a G180A change and with the amino acid changes indicated in bold (SEQ ID NO: 14);

FIG. 3A shows the DNA sequence of the ORF encoding subtilisin variant P23, with the start (ATG) and stop (TAA) codons underlined, the nucleotide changes indicated in bold, the associated codons underlined, and the region encoding the added histidine tag shown in bold italics (SEQ ID NO: 15);

FIG. 3B shows the DNA sequence of the ORF encoding subtilisin variant P23, with the start (ATG) and stop (TAA) codons underlined, the nucleotide changes indicated in bold, and the associated codons underlined (SEQ ID NO: 16);

FIG. 3C shows the polypeptide sequence of the subtilisin variant P23, with the amino acid changes indicated in bold and the added histidine tag shown in bold italics (SEQ ID NO: 17);

FIG. 3D shows the polypeptide sequence of the subtilisin variant P23, with the amino acid changes indicated in bold (SEQ ID NO: 18);

FIG. 4A is a photograph of heat inactivation of Subtilisin Carlsberg variant B24, with loss of activity at 50° C., 55° C., 60° C. and 65° C. within 10 minutes. Activity of protease was detected by spotting 7 μL of culture supernatant of SCK6 (pZY167::B24) that expresses the B24 protease;

FIG. 4B is a photograph of heat stability of wild type subtilisin Carlsberg, compared to Subtilisin Carlsberg variant B24, at 55° C., 60° C. and 65° C. over 40 minutes. The experiments were done with purified enzyme;

FIG. 5 is a gel showing the purification of the subtilisin B24 variant from culture supernatants;

FIG. 6 is a gel showing that the purified subtilisin B24 preparation does not contain detectable amounts of DNases;

FIG. 7 is a gel showing that the purified subtilisin B24 preparation does not contain detectable amounts of single stranded DNA nucleases;

FIG. 8 is a bar graph showing the heat inactivation of subtilisin B24 compared to proteinase K;

FIG. 9 is a gel showing subtilisin B24 inactivation of heat stable restriction enzymes Pvu I and Pvu II. Lane 1: DNA Ladder; Lane 2: PvuI treated with B24, then lambda DNA added; Lane 3: PvuI treated with heat inactivated B24, then lambda DNA added; Lane 4: PvuII treated with B24, then lambda DNA added; Lane 5: PvuII treated with heat inactivated B24, then lambda DNA added; Lane 6: B24 with lambda DNA;

FIG. 10 is a gel showing subtilisin B24 inactivation of heat stable restriction enzyme Pvu II at different time points. 10 μg of subtilisin B24 was mixed with 100 units of Pvu II (NEB) and incubated from 0 (lane 4), 10 (lane 5), 15 (lane 6), 20 (lane 7) or 30 minutes (lane 8) at 37° C. Control lanes show just B24 (lane 3) or just Pvu II (lane 2). Inactivation of B24 at 60° C. for 20 minute prior to adding it to Pvu II led to no digestion of Pvu II (lane 9);

FIG. 11A is a gel showing digestion of catalase with subtilisin B24 in the presence of SDS. Lane 1: Molecular weight ladder; Lane 2: Catalase+B24; Lane 3: Catalase+B24+0.2% SDS; Lane 4: Catalase+B24+0.4% SDS; Lane 5: Catalase+B24+0.8% SDS; Lane 6: Catalase+B24+1.0% SDS; Lane 7: Catalase+B24+1.5% SDS; Lane 8: Catalase+B24+2.0% SDS; Lane 9: Catalase+1.0% SDS;

FIG. 11B is a gel showing digestion of RNase A with Subtilisin B24 in the presence of SDS. Lane 1: Molecular weight ladder; Lane 2: RNase A+B24; Lane 3: RNase A+B24+0.2% SDS; Lane 4: RNase A+B24+0.4% SDS; Lane 5: RNase A+B24+0.8% SDS; Lane 6: RNase A+B24+1.0% SDS; Lane 7: RNase A+B24+1.5% SDS; Lane 8: RNase A+B24+2.0% SDS; Lane 9: RNase A+1.0% SDS;

FIG. 11C is a gel showing digestion of catalase with subtilisin B24 in the presence of Triton X-100. Lane 1: Molecular weight ladder; Lane 2: Catalase+B24; Lane 3: Catalase+B24+0.2% Triton X-100; Lane 4: Catalase+B24+0.4% Triton X-100; Lane 5: Catalase+B24+0.8% Triton X-100; Lane 6: Catalase+B24+1.0% Triton X-100; Lane 7: Catalase+B24+1.5% Triton X-100; Lane 8: Catalase+B24+2.0% Triton X-100; Lane 9: Catalase+1.0% Triton X-100+Heat inactivated B24; Lane 10: Catalase;

FIG. 11D is a gel showing digestion of RNase A with Subtilisin B24 in the presence of Triton X-100. Lane 1: Molecular weight ladder; Lane 2: RNase A+B24; Lane 3: RNase A+B24+0.2% Triton X-100; Lane 4: RNase A+B24+0.4% Triton X-100; Lane 5: RNase A+B24+0.8% Triton X-100; Lane 6: RNase A+B24+1.0% Triton X-100; Lane 7: RNase A+B24+1.5% Triton X-100; Lane 8: RNase A+B24+2.0% Triton X-100; Lane 9: RNase A+1.0% Triton X-100; Lane 10: RNase A;

FIG. 11E is a gel showing digestion of RNase A with Subtilisin B24 in the presence of CTAB. Lane 1: Molecular weight ladder; Lane 2: RNase A+B24; Lane 3: RNase A+B24+0.1× CTAB Buffer; Lane 4: RNase A+B24+0.2× CTAB Buffer; Lane 5: RNase A+B24+0.5× CTAB Buffer; Lane 6: RNase A+B24+1× CTAB Buffer; Lane 7: RNase A+B24+2× CTAB Buffer; Lane 8: RNase A; Lane 9: RNase A+2× CTAB Buffer; Lane 10: RNase A+Heated inactivated B24;

FIG. 12A is a gel showing the effect of heat treatment of subtilisin B24 and proteinase K on degradation of RNaseA; Lane 1: RNaseA; Lane 2: RNase A+B24; Lane 3: RNaseA+Proteinase K; Lane 4: RNaseA+Heat treated B24 (50° C. for 30 minutes); Lane 5: RNaseA+Heat treated proteinase K (50° C. for 30 minutes); Lane 6: RNaseA+Heat treated B24 (70° C. for 15 minutes); Lane 7: RNaseA+Heat treated proteinase K (70° C. for 15 minutes); Lane 8: RNaseA+Heat treated B24 (95° C. for 10 minutes); Lane 9: RNaseA+Heat treated proteinase K (95° C. for 10 minutes);

FIG. 12B is a gel showing the effect of heat treatment of subtilisin B24 and of proteinase K on degradation of the restriction enzyme Ase I; Lane 1: AseI; Lane 2: AseI+B24; Lane 3: AseI+Proteinase K; Lane 4: AseI+Heat treated B24 (50° C. for 30 minutes); Lane 5: AseI+Heat treated proteinase K (50° C. for 30 minutes); Lane 6: AseI+Heat treated B24 (70° C. for 15 minutes); Lane 7: AseI+Heat treated proteinase K (70° C. for 15 minutes); Lane 8: AseI+Heat treated B24 (95° C. for 10 minutes); Lane 9: AseI+Heat treated proteinase K (95° C. for 10 minutes); and

FIG. 13 is a graph showing the activity of subtilisin B24 and variants of subtilisin B24.

### DETAILED DESCRIPTION

The present disclosure provides, in part, variant subtilisin Carlsberg molecules and uses thereof. By “variant subtilisin Carlsberg molecules,” “subtilisin variant,” “variant polypeptides,” “variant,” or “variants,” as used herein, is meant the variant polypeptides including one or more of the mutations described herein, as well as nucleic acid molecules encoding such polypeptides. In some embodiments, the variant subtilisin Carlsberg polypeptides described herein are heat labile. In some embodiments, the variant subtilisin Carlsberg polypeptides described herein are not found in nature i.e., are “non-naturally occurring.”

In some embodiments, the present disclosure provides a nucleic acid molecule (for example, as set forth in SEQ ID NO: 1) encoding a variant subtilisin Carlsberg polypeptide, from Bacillus licheniformis, the nucleic acid molecule further including a mutation at one or more of the codons corresponding to positions K88, D180, N181, N265, L321, L339 or Q379. In some embodiments, the mutations may result in an amino acid change to one or more of K88N, D180G, N181Y, N265S, L321 F, L339M, or Q379P. In some embodiments, the present disclosure provides a nucleic acid molecule having at least 80% sequence identity, for example at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1) and including a mutation at one or more of the codons corresponding to positions K88, D180, N181, N265, L321, L339 or Q379 of a subtilisin Carlsberg polypeptide, from Bacillus licheniformis.

In some embodiments, the present disclosure provides a variant subtilisin Carlsberg polypeptide, from Bacillus licheniformis, as set out in GenBank No. AGN35600 (for example, as set forth in SEQ ID NO: 2), which further includes a mutation at one or more of positions K88, D180, N181, N265, L321, L339 or Q379. In some embodiments, the mutations may be one or more of K88N, D180G, N181Y, N265S, L321F, L339M, or Q379P. In some embodiments, the present disclosure provides a polypeptide having at least 80% sequence identity, for example at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2) and including a mutation at one or more of positions corresponding to K88, D180, N181, N265, L321, L339 or Q379 of a subtilisin Carlsberg polypeptide, from Bacillus licheniformis.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position K88. In some embodiments, the mutation may result in an amino acid change to K88N.

In some embodiments, the present disclosure provides a variant subtilisin Carlsberg polypeptide, from Bacillus licheniformis, as set out in GenBank No. AGN35600 (SEQ ID NO: 2), which further includes a mutation at K88. In some embodiments, the mutation may be K88N.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position D180. In some embodiments, the mutation may result in an amino acid change to D180G.

In some embodiments, the present disclosure provides a variant subtilisin Carlsberg polypeptide, from Bacillus licheniformis, as set out in GenBank No. AGN35600 (SEQ ID NO: 2), which further includes a mutation at D180. In some embodiments, the mutation may be D180G.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position N181. In some embodiments, the mutation may result in an amino acid change to N181Y.

In some embodiments, the present disclosure provides a variant subtilisin Carlsberg polypeptide, from Bacillus licheniformis, as set out in GenBank No. AGN35600 (SEQ ID NO: 2), which further includes a mutation at N181. In some embodiments, the mutation may be N181Y.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position N265. In some embodiments, the mutation may result in an amino acid change to N265S.

In some embodiments, the present disclosure provides a variant subtilisin Carlsberg polypeptide, from Bacillus licheniformis, as set out in GenBank No. AGN35600 (SEQ ID NO: 2), which further includes a mutation at N265. In some embodiments, the mutation may be N265S.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position L321. In some embodiments, the mutation may result in an amino acid change to L321F.

In some embodiments, the present disclosure provides a variant subtilisin Carlsberg polypeptide, from Bacillus licheniformis, as set out in GenBank No. AGN35600 (SEQ ID NO: 2), which further includes a mutation at L321. In some embodiments, the mutation may be L321F.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position L339. In some embodiments, the mutation may result in an amino acid change to L339M.

In some embodiments, the present disclosure provides a variant subtilisin Carlsberg polypeptide, from Bacillus licheniformis, as set out in GenBank No. AGN35600 (SEQ ID NO: 2), which further includes a mutation at L339. In some embodiments, the mutation may be L339M.

In some embodiments, the present disclosure provides a nucleic acid molecule (SEQ ID NO: 1) encoding a variant subtilisin Carlsberg polypeptide, from Bacillus licheniformis, the nucleic acid molecule further including a mutation at the codon corresponding to position Q379. In some embodiments, the mutation may result in an amino acid change to Q379P.

In some embodiments, the present disclosure provides a variant subtilisin Carlsberg polypeptide, from Bacillus licheniformis, as set out in GenBank No. AGN35600 (SEQ ID NO: 2), which further includes a mutation at Q379. In some embodiments, the mutation may be Q379P.

In some embodiments, the present disclosure provides a variant subtilisin Carlsberg polypeptide from Bacillus licheniformis, as set out in GenBank No. AGN35600 (SEQ ID NO: 2), which includes mutations at the following residues: L339 and Q379.

In some embodiments, the present disclosure provides a variant subtilisin Carlsberg polypeptide from Bacillus licheniformis, as set out in GenBank No. AGN35600 (SEQ ID NO: 2), which includes mutations at the following residues: D180 and L339.

In some embodiments, the present disclosure provides a variant subtilisin Carlsberg polypeptide from Bacillus licheniformis, as set out in GenBank No. AGN35600 (SEQ ID NO: 2), which includes mutations at the following residues: D180 and Q379.

In some embodiments, the present disclosure provides a variant subtilisin Carlsberg polypeptide (“Subtilisin Carlsberg variant B24,” “subtilisin B24,” “variant B24,” “B24 variant” or “B24”), from Bacillus licheniformis, as set out in GenBank No. AGN35600 (SEQ ID NO: 2), which further includes mutations at the following residues: D180, L339, and Q379.

In some embodiments, the Subtilisin Carlsberg variant B24 includes the following mutations: D180G, L339M, and Q379P (SEQ ID NO: 9 or 10).

In some embodiments, the Subtilisin Carlsberg variant B24 (variant B24-G180A) includes the following mutations: D180A, L339M, and Q379P (SEQ ID NO: 13 or 14).

In some embodiments, the Subtilisin Carlsberg variant (variant B24-G180D) includes the following mutations: L339M and Q379P (SEQ ID NO: 11 or 12).

In some embodiments, the present disclosure provides a nucleic acid molecule encoding the Subtilisin Carlsberg variant polypeptide (variant B24-G180D) which includes the following mutations: L339M and Q379P (SEQ ID NO: 11 or 12). In some embodiments, the present disclosure provides a nucleic acid molecule as set forth in SEQ ID NO: 5 or 6. In some embodiments, the present disclosure provides a polypeptide encoded by SEQ ID NO: 5 or 6.

In some embodiments, the present disclosure provides a nucleic acid molecule encoding the Subtilisin Carlsberg variant B24 polypeptide which includes mutations at the following residues: D180, L339, and Q379.

In some embodiments, the present disclosure provides a nucleic acid molecule encoding the Subtilisin Carlsberg variant B24 polypeptide which includes the following mutations: D180G, L339M, and Q379P (SEQ ID NO: 9 or 10). In some embodiments, the present disclosure provides a nucleic acid molecule as set forth in SEQ ID NO: 3 or 4. In some embodiments, the present disclosure provides a polypeptide encoded by SEQ ID NO: 3 or 4.

In some embodiments, the present disclosure provides a nucleic acid molecule encoding the Subtilisin Carlsberg variant B24 polypeptide (variant B24-G180A) which includes the following mutations: D180A, L339M, and Q379P (SEQ ID NO: 13 or 14). In some embodiments, the present disclosure provides a nucleic acid molecule as set forth in SEQ ID NO: 7 or 8. In some embodiments, the present disclosure provides a polypeptide encoded by SEQ ID NO: 7 or 8.

In some embodiments, the present disclosure provides a variant subtilisin Carlsberg polypeptide from Bacillus licheniformis, as set out in GenBank No. AGN35600 (SEQ ID NO: 2), which further includes mutations at the following residues: K88, D180, N181, N265, and L321 or combinations thereof, such as K88 and D180; K88 and N181; K88 and N265; K88 and L321; D180 and N181; D180 and N265; D180 and L321; N181 and N265; N181 and L321; K88, D180, and N181; K88, D180, and N265; K88, D180, and L321; D180, N181, and N265; D180, N265, and L321; N181, N265, and L321; K88, D180, N181, N265, and L321; etc.

In some embodiments, the present disclosure provides a variant subtilisin Carlsberg polypeptide (“Subtilisin Carlsberg variant P23,” “subtilisin P23,” “variant P23,” or “P23”), from Bacillus licheniformis, as set out in GenBank No. AGN35600 (SEQ ID NO: 2), which further includes mutations at the following residues: K88, D180, N181, N265, and L321.

In some embodiments, the Subtilisin Carlsberg variant P23 includes the following mutations: K88N, D180G, N181Y, N265S, and L321F (SEQ ID NO: 17 or 18).

In some embodiments, the present disclosure provides a nucleic acid molecule encoding the Subtilisin Carlsberg variant P23 polypeptide which includes mutations at the following residues: K88, D180, N181, N265, and L321.

In some embodiments, the present disclosure provides a nucleic acid molecule encoding the Subtilisin Carlsberg variant P23 polypeptide which includes the following mutations: K88N, D180G, N181Y, N265S, and L321F (SEQ ID NO: 17 or 18). In some embodiments, the present disclosure provides a nucleic acid molecule as set forth in SEQ ID NO: 15 or 16. In some embodiments, the present disclosure provides a polypeptide encoded by SEQ ID NO: 15 or 16.

It is well known in the art that some modifications and changes can be made in the structure of a polypeptide without substantially altering the biological function of that peptide, to obtain a biologically equivalent polypeptide. In one aspect of the invention, variant subtilisin Carlsberg polypeptides also extend to biologically equivalent peptides that differ from a portion of the sequence of the variant subtilisin Carlsberg polypeptides by conservative amino acid substitutions that retain protease activity but do not affect the other properties (e.g., heat lability and/or lack of cold adaptation) of the variant subtilisin Carlsberg polypeptides described herein. Accordingly, in some embodiments, the present disclosure provides variant subtilisin Carlsberg polypeptides and nucleic acid molecules that include mutations at positions K88, D180, N181, N265, L321, L339 or Q379 and may further include conservative substitutions or other mutations, where the conservative substitutions or other mutations retain protease activity but do not affect the other properties (e.g., heat lability and/or lack of cold adaptation). Such polypeptides and nucleic acid molecules may have at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 2, as appropriate.

As used herein, the term “conserved amino acid substitutions” refers to the substitution of one amino acid for another at a given location in a polypeptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the polypeptide by routine testing.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Ornithine.

In some embodiments, conserved amino acid substitutions may be made where an amino acid residue is substituted for another having a similar hydrophilicity value (e.g., within a value of plus or minus 2.0, or plus or minus 1.5, or plus or minus 1.0, or plus or minus 0.5), where the following may be an amino acid having a hydropathic index of about −1.6 such as Tyr (−1.3) or Pro (−1.6) are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (O); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conservative amino acid substitutions may be made where an amino acid residue is substituted for another having a similar hydropathic index (e.g., within a value of plus or minus 2.0, or plus or minus 1.5, or plus or minus 1.0, or plus or minus 0.5). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conservative amino acid substitutions may be made using publicly available families of similarity matrices (Altschul, S. F. 1991. “Amino acid substitution matrices from an information theoretic perspective.” Journal of Molecular Biology, 219: 555-665; Dayhoff, M. O., Schwartz, R. M., Orcutt, B. C. 1978. “A model of evolutionary change in proteins.” In “Atlas of Protein Sequence and Structure” 5(3) M. O. Dayhoff (ed.), 345-352, National Biomedical Research Foundation, Washington; States, D. J., Gish, W., Altschul, S. F. 1991. “Improved Sensitivity of Nucleic Acid Database Search Using Application-Specific Scoring Matrices” Methods: A companion to Methods in Enzymology 3(1): 66-77; Steven Henikoff and Jorja G. Henikoff. 1992 “Amino acid substitution matrices from protein blocks.” Proc. Natl. Acad. Sci. USA. 89(biochemistry): 10915-10919; M. S. Johnson and J. P. Overington. 1993. “A Structural Basis of Sequence Comparisons: An evaluation of scoring methodologies.” Journal of Molecular Biology. 233: 716-738. Steven Henikoff and Jorja G. Henikoff. 1993. “Performance Evaluation of Amino Acid Substitution Matrices.” Proteins: Structure, Function, and Genetics. 17: 49-61; Karlin, S. and Altschul, S. F. 1990. “Methods for assessing the statistical significance of molecular sequence features by using general scoring schemes” Proc. Natl. Acad. Sci. USA. 87: 2264-2268.) The PAM matrix is based upon counts derived from an evolutionary model, while the Blosum matrix uses counts derived from highly conserved blocks within an alignment. A similarity score of above zero in either of the PAM or Blosum matrices may be used to make conservative amino acid substitutions.

In alternative embodiments, conservative amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (J. Mol. Bio. 179:125-142, 184). Genetically encoded hydrophobic amino acids include Gly, Ala, Phe, Val, Leu, Ile, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₆-C₂₀ aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Trp, while non-genetically encoded aromatic amino acids include phenylglycine, 2-napthylalanine, beta-2-thienylalanine, 1,2,3,4-tetrahydro-isoquinoline-3-carboxylic acid, 4-chlorophenylalanine, 2-fluorophenylalanine3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids ornithine, 2,3,-diaminopropionic acid, 2,4-diaminobutyric acid, and homoarginine.

It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-im-benzylhistidine for the imidazole nitrogen of histidine. Peptide analogues also include amino acids that have been chemically altered, for example, by methylation, by amidation of the C-terminal amino acid by an alkylamine such as ethylamine, ethanolamine, or ethylene diamine, or acylation or methylation of an amino acid side chain (such as acylation of the epsilon amino group of lysine). Peptide analogues can also include replacement of the amide linkage in the peptide with a substituted amide (for example, groups of the formula —C(O)—NR, where R is (C₁-C₆) alkyl, (C₁-C₆) alkenyl, (C₁-C₆) alkynyl, substituted (C₁-C₆) alkyl, substituted (C₁-C₆) alkenyl, or substituted (C₁-C₆) alkynyl) or isostere of an amide linkage (for example, —CH₂NH—, —CH₂S, —CH₂CH₂—, —CH═CH— (cis and trans), —C(O)CH₂—, —CH(OH)CH₂—, or —CH₂SO—). The nucleic acid sequences, as described herein, may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The nucleic acid or polypeptide molecules, as described herein, may be “isolated” i.e., separated from the components that naturally accompany it. Typically, a molecule is isolated when it is at least 70%, 75%, 80%, or 85%, or over 90%, 95%, or 99% by weight, of the total material in a sample. Thus, for example, a polypeptide that is chemically synthesised, produced by recombinant technology, isolated by known purification techniques or as described herein, will be generally be substantially free from its naturally associated components. An isolated molecule can be obtained, for example, by extraction from a natural source that has been subjected to, for example, mutagenesis techniques as described herein or known in the art; by expression of a recombinant nucleic acid molecule encoding a polypeptide compound; or by chemical synthesis. The degree of isolation or purity can be measured using any appropriate method such as column chromatography, gel electrophoresis, HPLC, etc. A nucleic acid molecule is “isolated” when it is not immediately contiguous with (i.e., covalently linked to) the coding sequences with which it is normally contiguous in the naturally occurring genome of the organism from which the DNA of the invention is derived. Therefore, an “isolated” nucleic acid molecule is intended to mean a nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid molecule may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. The term therefore includes, e.g., a recombinant nucleic acid incorporated into a vector, such as an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. Preferably, an isolated nucleic acid comprises at least about 70%, 80%, 90%, 95%, or 99% (on a molar basis) of all macromolecular species present. Thus, an isolated nucleic acid molecule can include a nucleic acid molecule which is synthesized chemically or by recombinant means. Recombinant DNA contained in a vector are included in the definition of “isolated” as used herein. Also, isolated nucleic acid molecules include recombinant DNA molecules in heterologous host cells, as well as partially or substantially purified DNA molecules in solution.

Polypeptides, peptides or analogues thereof can be synthesised by standard chemical techniques, for example, by automated synthesis using solution or solid phase synthesis methodology. Automated peptide synthesisers are commercially available and use techniques well known in the art. Polypeptides, peptides or analogues thereof can also be prepared using recombinant DNA technology using standard methods such as those described in, for example, Sambrook, et al. (Molecular Cloning: A Laboratory Manual. 2^{nd} ed., Cold Spring Harbor Laboratory, Cold Spring Harbor Laboratory Press, Cold Spring Harbor, N.Y., 1989) or Ausubel et al. (Current Protocols in Molecular Biology, John Wiley & Sons, 1994).

In some embodiments, the variant polypeptides may include additional sequences that, for example, assist in purification. For example, the variant polypeptides may include polyhistidine tags, epitope tags, FLAG tags, or GST sequences, as described herein or known in the art.

The variant polypeptides (such as variant B24 or p23) can be prepared employing standard methods in molecular biology and biochemistry. For example, a plasmid or suitable vector expressing a variant polypeptide (an “expression vector”) can be transformed into a suitable host cell. A suitable vector can include, without limitation, a plasmid, a cosmid, a phage, a virus, a bacterial artificial chromosome (BAC), a yeast artificial chromosome (YAC), etc., into which a nucleic acid sequence, as described herein, can be inserted such that a variant polypeptide, as described herein, is expressed by a suitable host cell. The vector may include regulatory sequences, such as a promoter, enhancer, etc. and/or selectable markers, such as those that confer antibiotic resistance. Suitable vectors are commercially available or known in the art.

Suitable host cells can include, without limitation, bacterial (e.g., E. coli, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptomyces achromogenes, Streptomyces avermitilis, Streptomyces coelicolor, Streptomyces griseus, Streptomyces lividans, Salmonella typhimurium, etc.), fungal (e.g., Saccharomyces cerevisiae, Pichia pastoris, or Neurospora crassa), plant, insect (e.g., Drosophila or Spodopterafrugiperda) or other animal cells, as long as they are capable of expressing functional (e.g., heat labile) variant polypeptides as described herein. Host cells can be cultured in conventional nutrient media modified as appropriate for activating promoters, selecting transformants, or expressing the variant polypeptides as known in the art or described herein.

In some embodiments, a strain of Bacillus subtilis that has been engineered to delete five of the seven genes that encode secreted proteases can be used. Such a strain can be obtained, for example, from the Bacillus Genetic Stock Center (BGSC code 1A1097; Doi R H, He X S, McCready P, Bakheit N. Bacillus subtilis: a model system for heterologous gene expression. in Applications of Enzyme Biotechnology. eds Kelly J W, Baldwin T O (Plenum Press, New York, N.Y.), pp 261-272). In such a strain the variant polypeptide is the dominant secreted protease and one of the few secreted proteins that is found in the culture supernatant. The variant polypeptide-expressing strain is grown at 31° C. in LB broth (10 gm/L tryptone; 5 gm/L yeast extract; 5 gm/L NaCl) to late stationary phase. The cells in the culture are removed by centrifugation or tangential flow filtration. If centrifugation is used the culture supernatant is cleared by filtration through a 0.22 μm filter. The cleared supernatant is applied to a column containing HIS-Select™ Nickel Affinity Gel-SIGMA. Before the cleared supernatant is applied the affinity gel is washed with 2 column volumes of deionized water to remove the 20% ethanol storage buffer and then equilibrated with 3 column volumes of equilibration buffer (100 mM HEPES [pH 7.5], 10 mM imidazole, 100 mM NaCl, 10 mM CaCl₂). The clarified crude lysate is loaded onto the column at a flow rate of ˜2 column volumes/hour. The flow-through is collected in fractions and each fraction is collected for 5 minutes. The column is then washed with wash buffer (100 mM HEPES [pH _{7.5],} 10 mM imidazole, 100 mM NaCl, 10 mM CaCl₂) at a flow rate of ˜10 column volumes/hour until the A280 reaches the same A280 as the wash buffer. The His-tagged B24 protease is eluted from the column using elution buffer (100 mM HEPES [pH 7.5], 150 mM imidazole, 100 mM NaCl, 10 mM CaCl₂) at a flow rate of 3 column volumes/hour until the A280 reaches the same A280 as the elution buffer. Fractions containing protease activity are pooled, dialyzed to remove the imidazole and lyophilized.

In general, a variant polypeptide according to the present disclosure is heat-labile. By “heat-labile” is meant a polypeptide that exhibits substantial loss of activity, for example, protease activity, upon exposure to temperatures over about 50° C. for at least 10 minutes. In some embodiments, a heat-labile variant polypeptide as described herein exhibits substantial loss of activity at temperatures over about 50° C., such as about 55° C., 60° C., 65° C., 70° C. or 80° C., or any value between about 50° C. and about 80° C., or over about 80° C., for at least about 10 minutes, such as about 15, 20, 30, or 45 minutes, or any value between about 10 minute or about 45 minutes, or more. In some embodiments, a heat-labile polypeptide as described herein (such as the B24 variant at a concentration of 100 μg/ml (micrograms/nil)) exhibits >95% loss of its activity in 45 minutes upon heating to 50° C.; or in 30 minutes upon heating to 60° C.; or in 20 min upon heating to 65° C.; or in 15 minutes upon heating to 70° C.; or in 10 min upon heating to 80° C.

In some embodiments, a heat-labile polypeptide according to the present disclosure may be a polypeptide that exhibits substantial loss of activity upon exposure to temperatures at which other molecules (e.g., DNA, RNA, polypeptides, small molecules) are stable and/or do not exhibit loss of activity. For example, in some embodiments, a heat-labile polypeptide according to the present disclosure may exhibit loss of activity at a temperature sufficient to preserve DNA in a double-stranded form.

It is to be understood that full or 100% loss of activity is not required and that parameters such as the pH, target substrate, and the concentration of the variant polypeptide can affect its activity. Accordingly, in some embodiments, substantial loss of activity can be determined according to standard techniques, depending on such parameters. In alternative embodiments, a substantial loss of activity can include about 50% to about 100% loss of activity, or any value therebetween, such as about 55%, 60%, 65%, 70%, 75%, 80%, 85%, 90%, 95%, or 99% loss of activity.

In some embodiments, a polypeptide according to the present disclosure exhibits optimal activity at temperatures of about 20° C. to about 40° C., or any value therebetween, such as about 20° C., 25° C., 30° C., 35° C., 37° C., 40° C., etc. In some embodiments, a polypeptides according to the present disclosure exhibits optimal activity at about 37° C.

In general, a variant polypeptide according to the present disclosure may be used in any application in which heat-lability is useful. For example, variant polypeptide according to the present disclosure may be used to digest or degrade a target polypeptide present in a sample, under conditions suitable for protease activity of the variant polypeptide. After digestion or degradation of the target polypeptide has proceeded to the extent determined to be sufficient under the specific circumstances, the variant polypeptide may be inactivated by increasing the temperature of the sample. A “target polypeptide” may include, without limitation, an enzyme, a nuclease, or any other protein, polypeptide, or proteinaceous material that needs to be removed. By “removal” of a target polypeptide is meant removal, reduction and/or inactivation of the target polypeptide by, for example, digestion or degradation by a protease. It is to be understood that 100% removal is not always required and therefore a target polypeptide may be considered sufficiently removed if the amount or activity of the polypeptide is reduced by at least 10%, 20%, 30%, 40%, 50%, 60%, 70%, 80%, 90%, 95%, or 99%, after treatment with a protease, compared to its amount or activity prior to treatment. A sample may be any material from which removal of a target polypeptide is desired, for example, a molecular biology sample, a clinical sample, a diagnostic sample, a forensic sample, etc.

In some embodiments, a variant polypeptide according to the present disclosure may be used to digest molecular biology enzymes and/or other proteins in a DNA manipulation technique or any technique that requires the removal of a protein, followed by a moderate heat inactivation step.

In some embodiments, a variant polypeptide according to the present disclosure may be used to digest molecular biology enzymes, for example, heat resistant enzymes such as Taq polymerase and the heat resistant restriction enzyme Pvu II.

In some embodiments, a variant polypeptide according to the present disclosure may be used to remove contaminating DNA degrading nucleases, DNA modifying enzymes and/or other proteins from a preparation of plasmid DNA isolated from Escherichia coli, such as a standard mini-preparation.

In some embodiments, a variant polypeptide according to the present disclosure may be used to remove contaminating DNA degrading nucleases, DNA modifying enzymes and/or other proteins from a preparation of chromosomal DNA isolated from, for example, a microbial, plant, and/or animal cell.

In some embodiments, a variant polypeptide according to the present disclosure may be used to remove contaminating DNA degrading nucleases, DNA modifying enzymes and/or other proteins from a preparation of mitochondrial DNA isolated from an animal cell.

In some embodiments, a variant polypeptide according to the present disclosure may be used to remove contaminating proteins from a forensic sample or a clinical and/or diagnostic sample. For example, a variant polypeptide according to the present disclosure may be used prior to probing the sample with an antibody, such as in a drug test, or prior to amplifying DNA, such as in a paternity test.

In some embodiments, a variant polypeptide according to the present disclosure may be used to remove RNA degrading nucleases and/or RNA modifying enzymes from a preparation of RNA.

In some embodiments, a variant polypeptide according to the present disclosure may be used in a process of making cDNA from mRNA. For example, a preparation of mixed nucleic acids may be treated with a DNase to remove contaminating DNA. A variant polypeptide according to the present disclosure may then be added to remove the DNase, after which the polypeptide according to the present disclosure may be inactivated by exposing it to a temperature at which it exhibits heat lability. The cDNA may then be produced from the mRNA.

In some embodiments, a variant polypeptide according to the present disclosure may be used to remove a restriction enzyme (e.g., AvrII, BamHI, BgIII, DraIII, HpaI, KpnI, MfeI, PstI, PvuII, Tsp509I, etc.; see the New England Biolabs section Tools & Resources section relating to Heat Inactivation at https[://]www[.]neb[.]com/tools-and-resources/usage-guidelines/heat-inactivation, which lists a number of restriction enzymes that cannot be heat-inactivated) following digestion of DNA followed by heat inactivation of the polypeptide according to the present disclosure by exposing it to a temperature at which it exhibits heat lability. In some embodiments, a variant polypeptide according to the present disclosure may be used to remove a restriction or other enzyme which cannot be inactivated using standard heat inactivation techniques.

In some embodiments, a variant polypeptide according to the present disclosure may be used to remove a DNA modifying enzyme, such as alkaline phosphatase or T4 DNA kinase, followed by heat inactivation of the polypeptide according to the present disclosure by exposing it to a temperature at which it exhibits heat lability.

In some embodiments, a variant polypeptide according to the present disclosure may be used in the purification of proteins aggregated in inclusion bodies to remove contaminating proteins, followed by heat inactivation of the polypeptide according to the present disclosure by exposing it to a temperature at which it exhibits heat lability.

In some embodiments, a variant polypeptide according to the present disclosure may be used in the purification of a carbohydrate to remove contaminating proteins, followed by heat inactivation of the polypeptide according to the present disclosure by exposing it to a temperature at which it exhibits heat lability, and then modification of the carbohydrate with an enzyme.

In some embodiments, a variant polypeptide according to the present disclosure may be used in the purification of a lipid to remove contaminating proteins, followed by heat inactivation of the polypeptide according to the present disclosure by exposing it to a temperature at which it exhibits heat lability, and then modification of the lipid with an enzyme.

In some embodiments, a variant polypeptide according to the present disclosure can be used in an automated process that involves successive steps that use enzymes, so that the enzymes used in one step are removed by action of the polypeptide according to the present disclosure (e.g., protease action), followed by heat inactivation of the polypeptide according to the present disclosure by exposing it to a temperature at which it exhibits heat lability, before the next step that involves the addition of another enzyme.

The variant polypeptides may be provided in a suitable amount, sufficient to achieve a desired level of protease activity, in a composition that may also include a suitable carrier. The carrier may be, without limitation, any component used in molecular biology, forensic, cleaning or other compositions.

The composition may be a cleaning composition for, for example, cleaning fabrics, carpets, dishes, etc. The composition may be in any suitable form, such as a liquid, gel, granule, cake, bar, paste, powder, or spray. In some embodiments, the cleaning composition may be a detergent composition, such as a laundry detergent or a dish detergent. It is to be understood that a cleaning composition may include other components, such as surfactants, chelating agents, bleach, fabric conditioners, polyols, lactic acid, boric acid, etc.

The present invention will be further illustrated in the following examples.

### EXAMPLES

Error-Prone PCR-Based Mutagenesis

Error-prone polymerase chain reaction (PCR)-based mutagenesis using B. subtilis as a host (Zhang, X-Z and Zhang Y-H. P. “Simple, fast and high-efficiency transformation system for directed evolution of cellulose in Bacillus subtilis, MicrobBiotechnol. 4(1): 98-105, 2011) was conducted and involved the following general steps: designing synthetic DNA and primers; generating a library of random DNA mutants with error prone PCR; multimerization of plasmids with overlap PCR; transforming the library into B. subtilis; and selecting for protein mutants.

Synthetic DNA and Primers

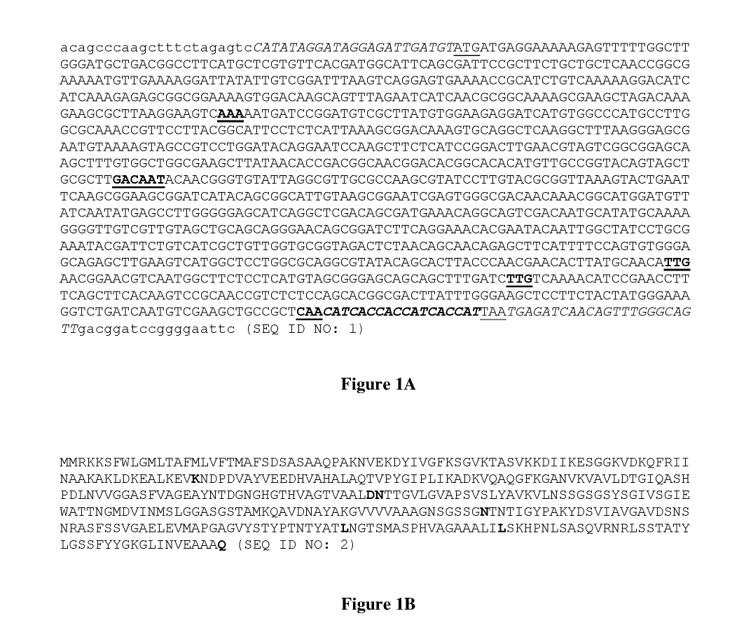

A synthetic gene with the sequence shown in FIG. 1 was obtained from Integrated DNA Technologies. The core of the sequence encodes a naturally occurring variation of subtilisin Carlsberg (subC) from the species Bacillus licheniformis (subtilisin Carlsberg AprE [Bacillus licheniformis 9945A]; GenBank: AGN35600). The 3′ end was altered to add a histidine tag before the stop codon. The 3′ and 5′ ends of the sequence contain sequence overlaps from the vector pZY167 (Zyprian E, Matzura H., Characterization of signals promoting gene expression on the Staphylococcus aureus plasmid pUB110 and development of a gram-positive expression vector system. DNA. 1986 June; 5(3):219-25; PMID: 3013549) to allow for overlap PCR of the subtilisin gene with the plasmid.

The primers for amplifying the synthetic gene for cloning and subsequent error prone PCR were:

(SEQ ID NO: 19)
SubC-F: TCAGCCCAAGCTTTCTAGAGTCCA,
and
(SEQ ID NO: 20)
SubC-R: GAATTCCCCGGATCCGTCAAC.

The primers for amplifying the vector pZY167 for creating plasmid multimers with the subtilisin gene via overlap PCR were:

167-S.CtoL:
(SEQ ID NO: 21)
ATCAATCTCCTATCCTATATGGACTCTAGAAAGCTTGGGCTGA
and
167-S.CtoR:
(SEQ ID NO: 22)
TGAGATCAACAGTTTGGGCAGTTGACGGATCCGGGGAATTC.

The synthetic gene was amplified by PCR and then joined to the vector pZY167 to generate the plasmid pZY167::subC.

Error Prone PCR

A randomly mutated library of subC was generated by error-prone PCR as follows. First, the synthetic subtilisin Carlsberg gene was amplified using the PrimeSTAR GXL polymerase. 2 μl of that reaction was used as template in a 100 μl PCR reaction using Mutazyme II polymerase (Agilent Technologies) using the manufacturer's protocol. The PCR amplicon was purified using a Nucleospin PCR cleanup column.

Plasmid Multimerization by Overlap PCR

The plasmid pZY167 was linearized by inverse PCR using the primers 167-S.CtoL and 167-S.CtoR. The linearized plasmid and error-prone PCR reaction products of subC were purified using a Nucleospin PCR cleanup column. The multimerization process was done according to Zhang and Zhang, supra, using template pZY167 at 0.15 ng/μl, the error-prone PCR product of the synthetic subtilisin C gene at 5 ng/μl, and PrimeSTAR GXL DNA polymerase to carry out the amplification.

Preparation and Transformation of B. subtilis Cells

The B. subtilis SCK6 strain was inoculated into 3 ml of LB medium with 1 μg/ml⁻¹ erythromycin in a test tube. The cells were cultivated at 37° C. with shaking at 200 rpm overnight (about 14 hours). The culture was then diluted to 1.0 A₆₀₀ in a fresh LB medium containing 1% (w/v) xylose and then grown for 2 hours. The resulting supercompetent cell culture was ready to be transformed. One microlitre of the PCR product containing plasmid multimers was mixed with 100 μl of the supercompetent cells in a plastic test tube and cultivated at 37° C. with shaking at 200 rpm for 90 min, then 100 μl aliquots were plated onto LB agar petri plates supplemented with 25 μg/ml of kanamycin sulfate and 1% skim milk powder (EMD Millipore).

Screening Colonies for Temperature Sensitive Protease

Out of approximately 800 colonies grown at 30° C., about 2% had active protease as determined by zones of clearing around the colonies. The milk plates were 1% skim milk powder (EMD Millipore) with 1.5% agar (Difco). Zones of clearing were circles of transparency created when active protease digests the milk, which creates a cloudy appearance to the plates. The 2% of clones with active protease were grown in 3 ml of LB broth with 25 μg/ml of kanamycin sulfate. Cells were pelleted, the supernatant containing unpurified protease was collected, and 50 μl aliquots were heat treated at 60° C. for 10 minutes; duplicate aliquots were kept at room temperature. The room temperature and the 60° C.-treated aliquots were screened for their ability to produce zones of clearing on milk agar plates. Subtilisin variants that could not be heat inactivated were discarded. Approximately 20% of these variants were heat inactivated, in that they did not produce a zone of clearing on milk plates after heat denaturation. Two of these variants (B24 and P23), with stable repeatable zones of clearing at room temperature and no activity after heat denaturation, were selected for sequencing and further characterization.

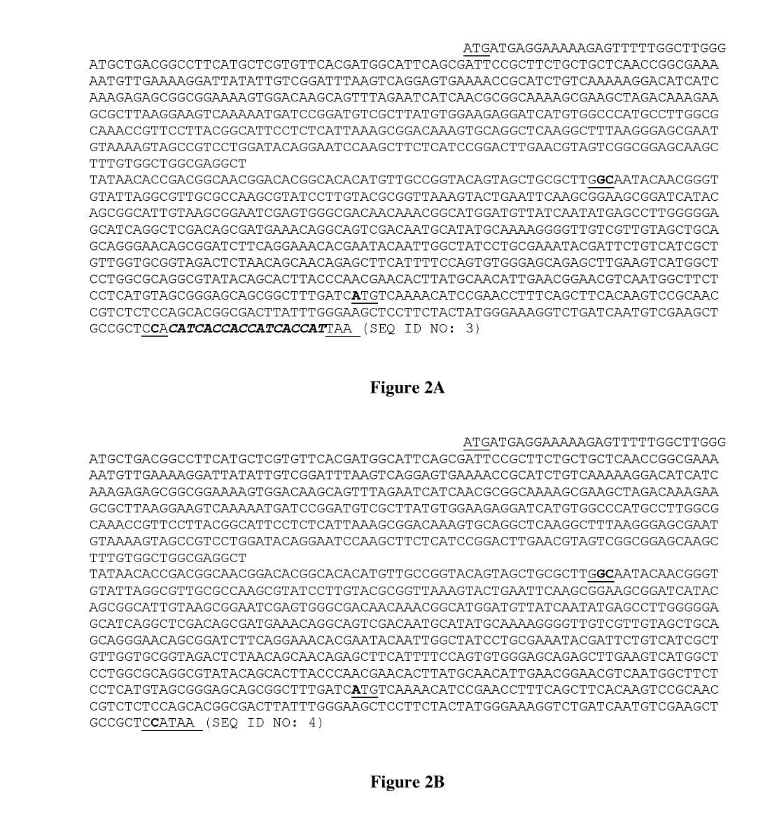

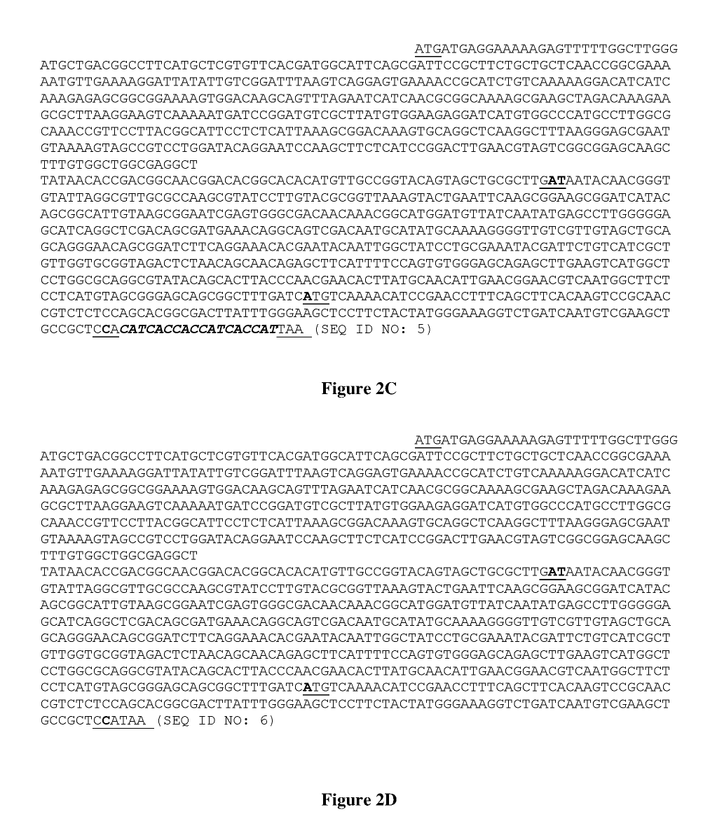

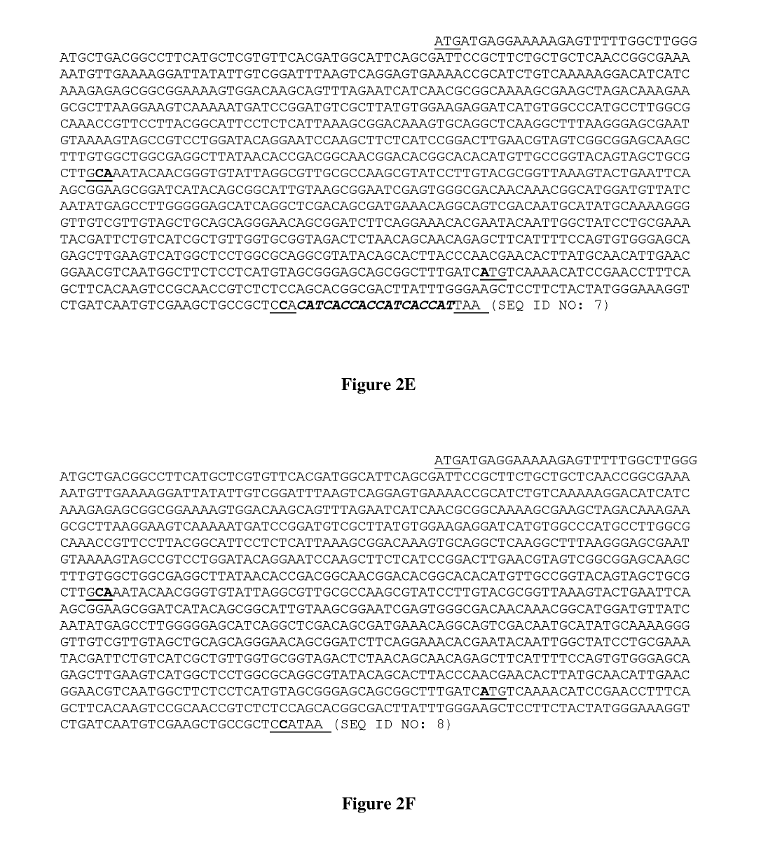

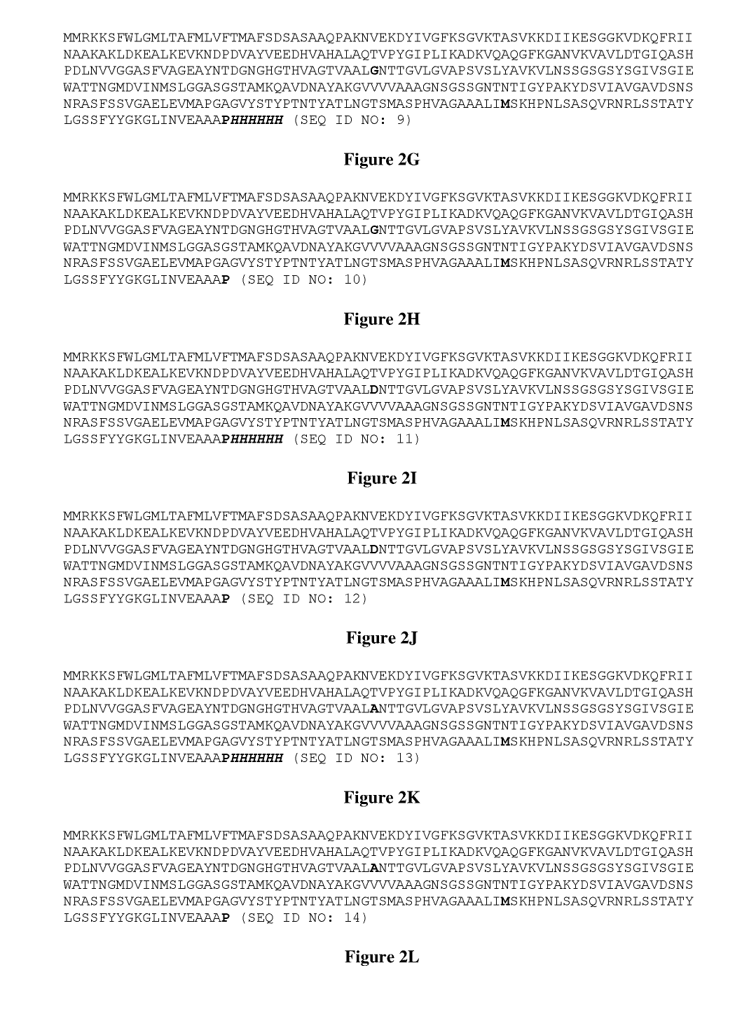

Subtilisin Carlsberg variant B24 had three amino acid changes at D180G, L339M, and Q379P. DNA sequences encoding heat-labile B24 variants of subtilisin are provided at FIGS. 2A-F.

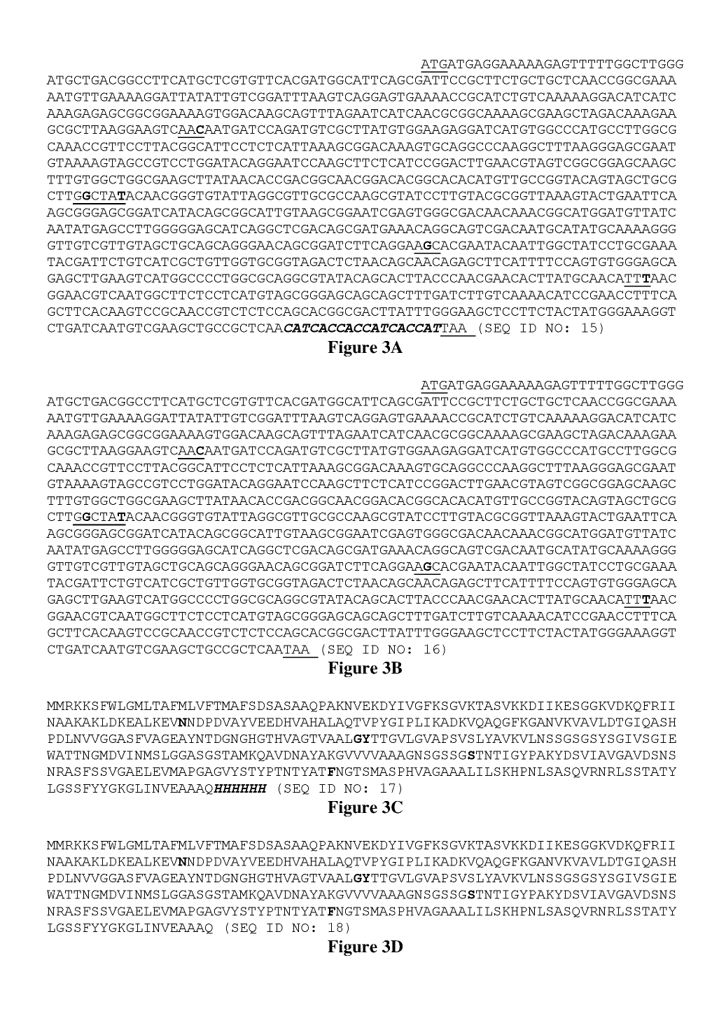

Subtilisin Carlsberg variant P23 had five amino acid changes at K88N, D180G, N181Y, N265S, and L321F. The DNA sequence encoding P23 is provided at FIGS. 3A-B.

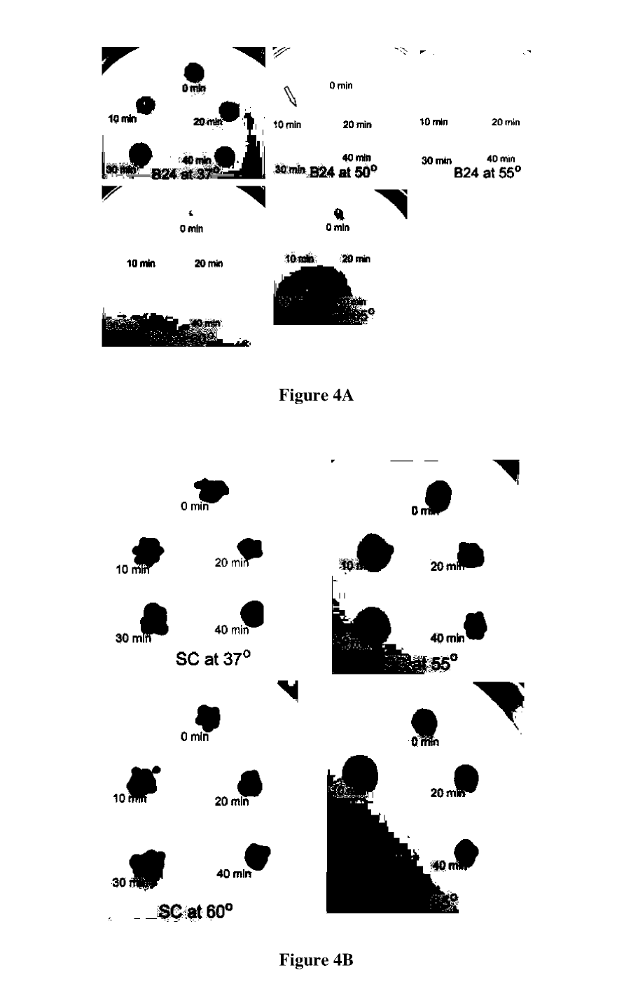

Both variants lost protease activity after incubation at 60° C. for 10 minutes, as determined by the ability to produce zones of clearing on milk agar plates. Variant B24 was further characterized through a time course assay which confirmed loss of activity after a 10-minute incubation 60° C. (FIG. 4).

Subtilisin B24 Purification from Culture Supernatants

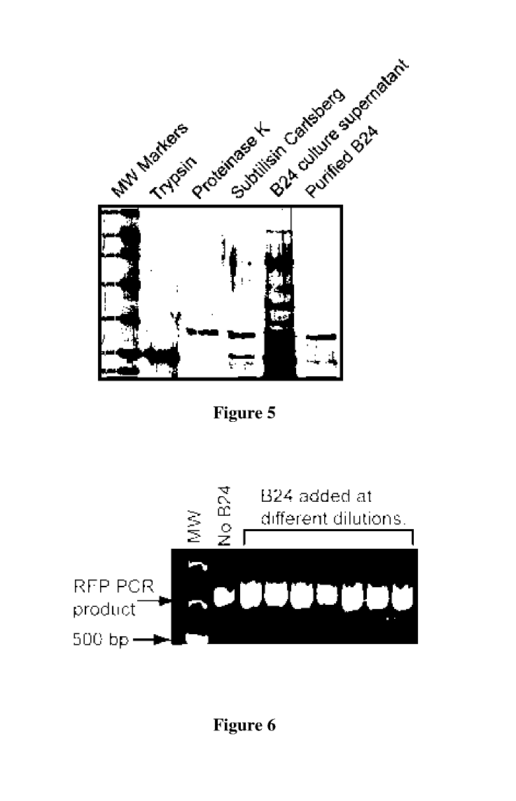

The Bacillus subtilis strain 1A1097 (Bacillus Genetic Stock Center, Columbus, Ohio) harboring a plasmid encoding the gene encoding B24 was streaked out for individual colonies on a LB agar plate containing 30 μg/mL of kanamycin and 1% dry milk. After 24 hour of incubation at 30° C. 10 isolated colonies showing zones of clearing of the milk opacity were chosen and used to inoculate 1 L of LB media containing 30 μg/mL of kanamycin. The culture was grown at 30° C. for 40 hours, the cells were removed via centrifugation and the subtilisin B24 was purified from the supernatant. The supernatant was further cleared by passing it through a 0.2 μm filter. An aliquot was saved to compare the purity before and after purification. The filtered supernatant was mixed 50:50 with equilibration buffer (20 mM HEPES, 300 mM NaCl, 10 mM imidazole, pH 7.5) and applied to a (nickle nitrilotriacetic acid (Ni-NTA) column (Sigma). The column was washed with wash buffer (20 mM HEPES, 300 mM NaCl, 25 mM imidazole, pH 7.4) to remove any loosely bound contaminating proteins. The B24 protease was eluted using elution buffer (20 mM HEPES, 300 mM NaCl, 200 mM imidazole, pH 7.4) into 5 mL fractions. From each fraction 5 μl was spotted onto milk agar (1% milk, 1.5% agar) to test for protease activity, as evidenced by the clearing of the opacity created by the milk. Fractions with protease activity were pooled, and purified B24 and supernatant were loaded onto a 10% SDS-PAGE gel along with commercial trypsin, proteinase K, and subtilisin Carlsberg (FIG. 5). The gel was run at 200V for 45 minutes and stained using a silver staining method. The B24 supernatant (lane 5) contained multiple bands while the purified B24 (lane 6) contained two bands with one of the bands at the correct ˜27 KDa molecular weight. The lower band is not considered contamination because the commercial subtilisin Carlsberg (lane 4) which is the parent protease, has the same banding pattern as the purified B24.

Purified subtilisin B24 was mixed with PCR amplicon of the red fluorescent protein gene (RFP) and was incubated for 1 h at 37° C. The incubated samples were loaded onto a 0.75% agarose gel containing Gel Red (FIG. 6). The gel was run at 70V for 2 hours. The bands were detected using UV light. The results indicated that incubation with subtilisin B24 did not lead to smearing or loss of PCR product. Therefore, purified subtilisin B24 did not contain DNases.

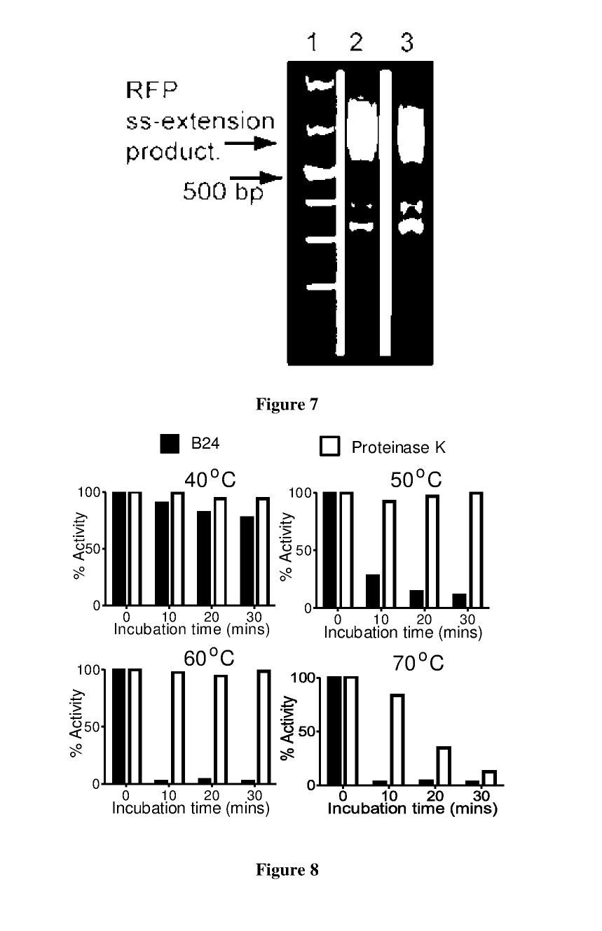

A single primer was used in 60 cycles of a mock PCR reaction to create single-stranded DNA. B24 was added to one sample (shown in lane 3) and incubated at 37° C. for 1 h (FIG. 7). The results indicated that incubation with subtilisin B24 did not lead to smearing or loss of single stranded DNA bands. Therefore, purified subtilisin B24 did not contain nucleases that attack single stranded DNA.

Heat Inactivation of Subtilisin B24 Compared to Proteinase K

Heat inactivation of subtilisin B24 was compared to proteinase K using a protease assay. The protease assay was conducted using the reagents supplied in a Pierce™ Fluorescent Protease Assay Kit (catalog number 23266). Solutions of the subtilisin B24 variant and proteinase K were made up to 1 mg/ml in a buffer of 25 mM Tris, 0.15M NaCl, at pH 7.2. To test sensitivity to heat,100 μl aliquots of each protease were incubated at temperatures of 40° C., 50° C., 60° C. and 70° C., for 0, 10, 20, and 30 minutes. After incubation, each of the protease solutions were transferred to wells in a 96 well plate and mixed with 100 μl of FTC-Casein solution provided with the assay kit. The assay plate was incubated at room temperature for 5 minutes and fluorescence read with a Molecular Devices Spectra Max M5 plate reader with the filters for excitation and emission set at 485 nm and 538 nm respectively.

The results indicated that subtilisin B24 was more heat labile than proteinase K (FIG. 8). More specifically, subtilisin B24 retained most of its activity at 40° C. for up to 30 minutes. This represents a typical digestion performed in molecular biology protocols, which are often done at 37° C. At 50° C., most of the subtilisin B24 activity was lost by 30 minutes. At 60° C. or 70° C. an incubation of 10 minutes or more eliminated all detectable protease activity from subtilisin B24. In contrast, proteinase K, the most commonly used laboratory protease, remains fully active after 30 minutes at 60° C., and still retains some activity after 30 minutes when incubated at 70° C. Thus, there are widely differing heat stability properties between subtilisin B24 and proteinase K. The instability of subtilisin B24 allows one to rapidly eliminate its protease activity at relatively moderate temperatures.

Subtilisin B24 Inactivation of Heat Stable Restriction Enzymes Pvu I and Pvu II

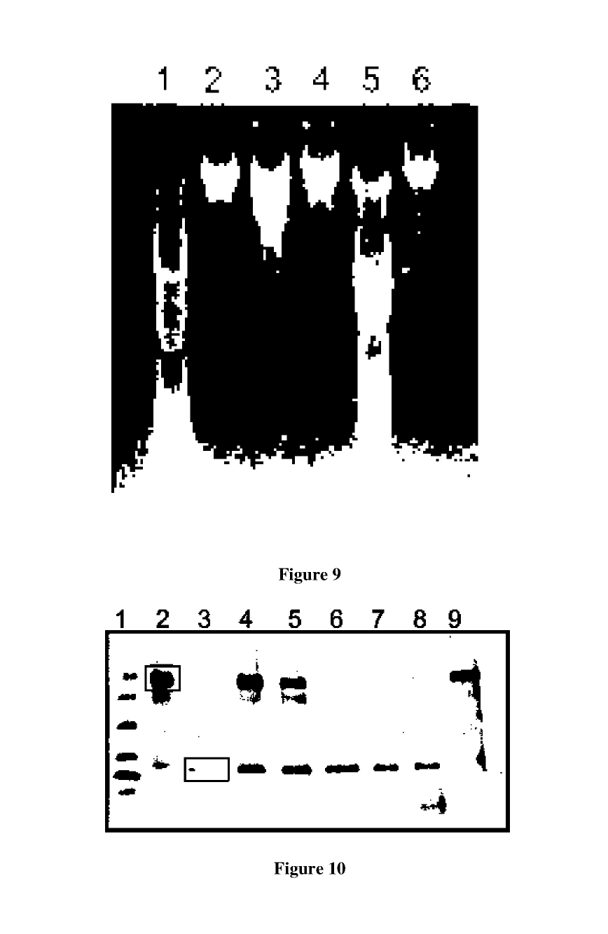

The restriction enzymes, Pvu I and Pvu II, are thermostable and are thus resistant to temperature inactivation, typically done at 65° C. or 80° C. The ability of subtilisin B24 to inactivate these enzymes was tested (FIG. 9). Briefly, the final reaction volume of the tests was 30 μl in 1× NEB 3.1 buffer (New England Biolabs, “NEB”). For lanes 2 and 4, 50 units of restriction enzymes Pvu I (NEB) and Pvu II (NEB) were incubated at 37° C. in the presence of 100 μg of subtilisin B24 for 1 hour. After this 1 hour treatment, subtilisin B24 was inactivated at 60° C. for 10 minutes. Then 2.5 μg of lambda DNA was added, and the tubes were incubated at 37° C. for another hour. For lanes 3 and 5, 100 μg of subtilisin B24 was heat inactivated at 60° C. for 10 minutes, then 50 units of restriction enzyme and 2.5 μg of DNA was added and incubated at 37° C. for an hour. Lastly, for lane 6, 100 μg of subtilisin B24 and 2.5 μg of DNA was incubated at 37° C. for 1 hour. 20 μL of each reaction was mixed with 5 μL of 6× loading dye and the full 25 μL was loaded onto a 1.5% agarose gel containing 2 μl of Gel Red™ (Biotium). Electrophoresis was carried out at 45V for 8 hours and then visualized using UV light. The results indicated that the subtilisin B24 variant digests and inactivates heat stable restrictions enzymes. More specifically, lanes 3 and 5 containing lamba DNA shows cutting of the lambda DNA as compared to the lambda DNA control containing no restriction enzyme in lane 6. Lanes 2 and 4 which contained restriction enzyme incubated with B24 (not heat treated) showed no lambda DNA digestion, which demonstrates that B24 inactivates heat-stable restriction enzymes Pvu I and Pvu II.

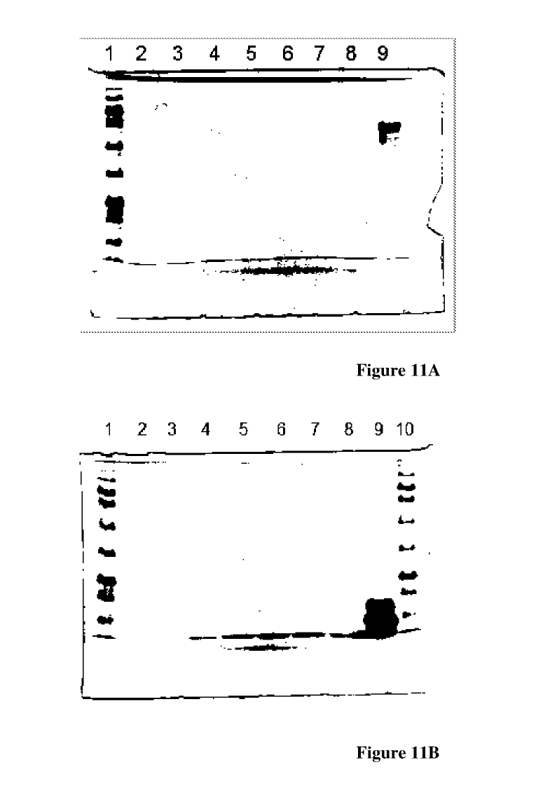

To visualize the digestion of Pvu II (FIG. 10), 10 μg of subtilisin B24 was mixed with 100 units of Pvu II (NEB) and incubated from 0 (lane 4), 10 (lane 5), 15 (lane 6), 20 (lane 7) or 30 minutes (lane 8) at 37° C. Control lanes show just B24 (lane 3) or just Pvu II (lane 2). Inactivation of B24 at 60° C. for 20 minute prior to adding it to Pvu II led to no digestion of Pvu II (lane 9).

Activity of Subtilisin B24 in the Presence of Detergents

Digestion of catalase and RNaseA (Bio Basic) with 10 μg of subtilisin B24 in the presence of varying concentrations of the detergents SDS, Triton X-100, and CTAB was determined. For each reaction condition, 10 μg of lyophilized catalase or RNase A and protease B24 were dissolved in 25 μL aliquots of 10 mM Tris-HCl, pH 8.0 containing the detergent. The 2× CTAB Buffer was 100 mM Tris-HCl, pH 8.0; 1.4M NaCl; 20 mM EDTA; 2% CTAB; 2% polyvinylpyrrolidone; 0.2% β-mercaptoethanol. The reactions were incubated at 37° C. for 1 hour. As a negative control, catalase and RNase A were incubated at 37° C. for 1 hour without subtilisin B24 in the presence of 1% detergent. After the incubation, 4× SDS loading dye was added to each sample and were incubated at 70° C. for 10 minutes, and then 20 μl was loaded onto a 12% SDS-PAGE gel. The gel was run at a constant current of 24 amps for 1 hour. The gel was Coomassie stained overnight.

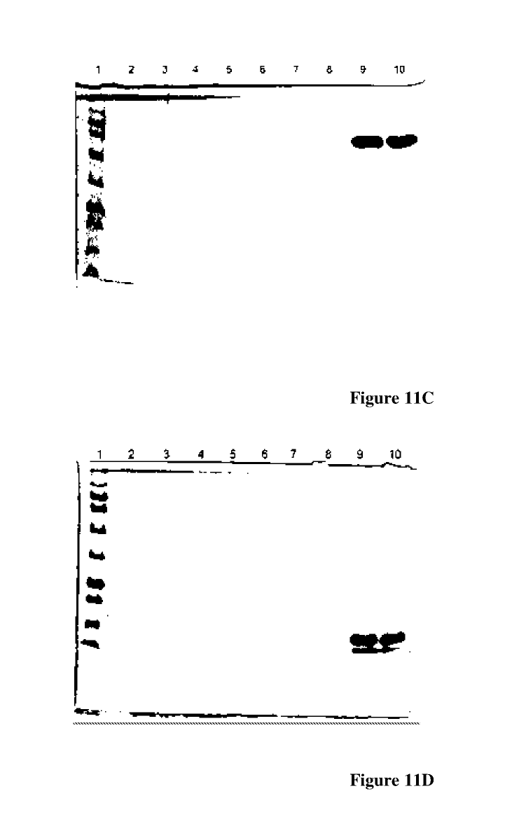

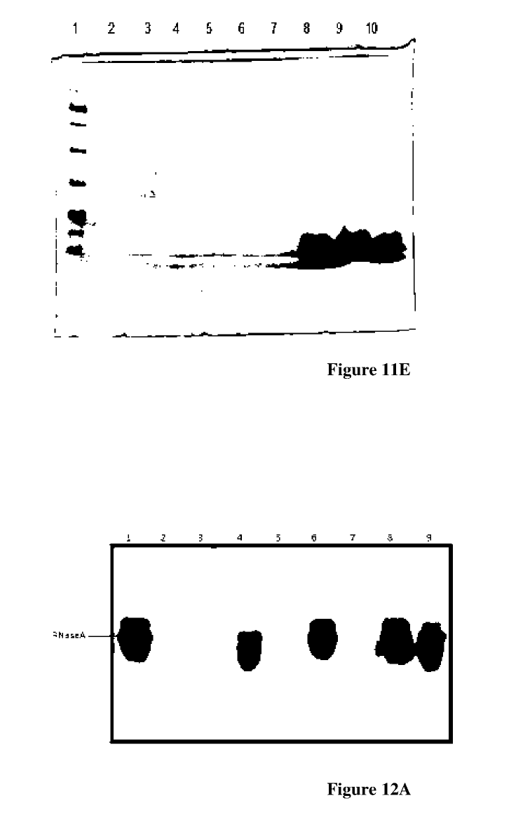

The results indicated that the subtilisin B24 variant can digest catalase (FIG. 11A) and RNAase (FIG. 11B) up to at least a 2.0% SDS concentration; catalase (FIG. 11C) and RNAase (FIG. 11D) up to at least a 2.0% Triton X-100 concentration; and RNAase (FIG. 11E) up to at least a 2% CTAB concentration.

Heat Treatment of Subtilisin B24 and Proteinase K

The subtilisin B24 variant and proteinase K (Bio Basic) were subjected to various exposure to heat. After the heat treatment, or mock treatment at room temperature, 5 μg of the subtilisin B24 variant or proteinase K (Biobasic) were mixed with 10 μg of RNaseA (Bio Basic, FIG. 12A) or the restriction enzyme Ase I (NEB, FIG. 12B) in a total of 20 μL of 10 mM Tris-HCl, pH 8.0, and incubated for 1 hour at 37° C. After the incubation, 4× SDS loading dye was added to each sample and were incubated at 70° C. for 10 minutes, and then 20 μL was loaded onto a 12% SDS-PAGE gel. The gel was run at a constant current of 24 amps for 1 hour. The gel was Coomassie stained overnight.

The results indicated that while both the subtilisin B24 variant and proteinase K can degrade RNaseA, the degradation activity of the subtilisin B24 variant is significantly reduced at 50° C. and eliminated at 70° C. in 15 minutes. By contrast, proteinase K activity is not eliminated at 70° C. for 15 min and the enzyme appears to retain some activity at 95° for 10 min.

The results also indicated that subtilisin B24 variant can digest Ase I in one hour at 37° C. while Proteinase K cannot.

Temperature Inactivation Subtilisin B24 and Variants of Subtilisin B24

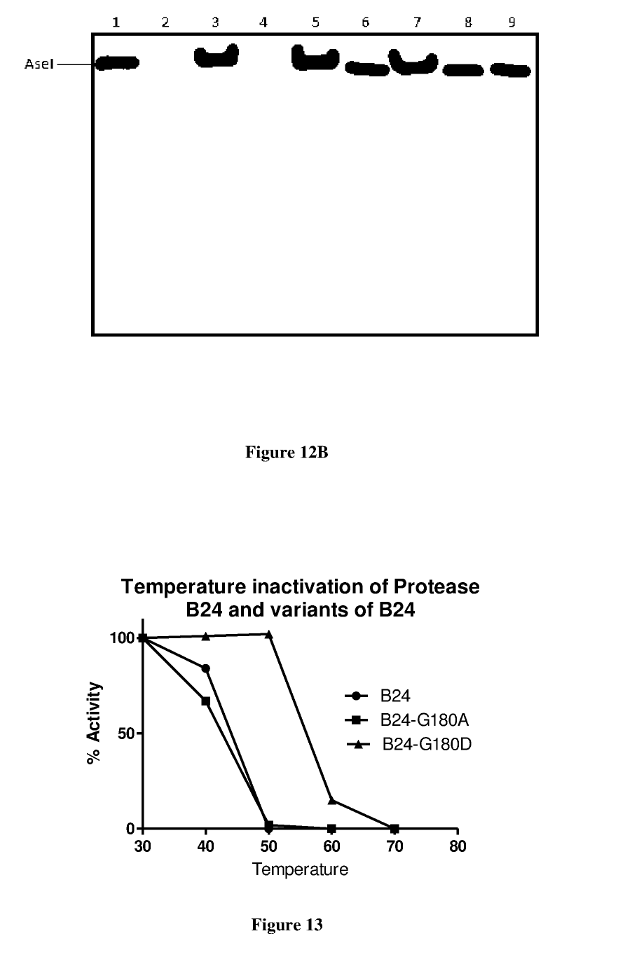

Amino acid residue 180 was changed to either an alanine (A) or an aspartic acid (D) residue, which is the amino acid residue found in the wild type parent subtilisin, in the context of the subtilisin B24 variant. B24G, B24-G180A, and B24-G180D were incubated between 30° C.-70° C. for 1 hour, as described herein. Protease activity was detected using a Pierce fluorescent protease assay kit and the percent activity was calculated using 30° C. activity as 100%. The G180A variant change exhibited a similar effect on protease thermostability as the subtilisin B24 D180D variant (FIG. 13). By contrast, the G180D variant change increased the stability considerably (FIG. 13).

All citations are hereby incorporated by reference.

As used herein the singular forms “a”, “and”, and “the” include plural referents unless the context clearly dictates otherwise. For example, “a variant” refers to one or more of such variants, “a cell” refers to a plurality of cells, while “the enzyme” includes a particular enzyme as well as other family member equivalents thereof as known to those skilled in the art.

The present invention has been described with regard to one or more embodiments. However, it will be apparent to persons skilled in the art that a number of variations and modifications can be made without departing from the scope of the invention as defined in the claims.

1. A variant subtilisin Carlsberg polypeptide, the polypeptide comprising a mutation at one or more of amino acids K88, D180, N181, N265, L321, L339 or Q379, or combinations thereof, wherein the polypeptide is heat labile.

2. The polypeptide of claim 1 comprising a mutation at amino acids D180, L339 and Q379; L339 and Q379; D180 and L339; D180 and Q379; or K88, D180, N181, N265, and L321.

3. The polypeptide of claim 1 wherein the mutation at K88 is K88N, the mutation at D180 is D180G or D180A, the mutation at N181 is N181Y, the mutation at N265 is N265S, the mutation at L321 is L321F, the mutation at L339 is L339M, or the mutation at Q379 is Q379P.

4. The polypeptide of claim 1 comprising the sequence set forth in any one of SEQ ID NOs: 9 to 14, 17 or 18.

5. A nucleic acid molecule encoding the polypeptide of claim 1.

6. A nucleic acid molecule comprising the sequence set forth in in any one of SEQ ID NOs: 3 to 8, 15 or 16.

7. An expression vector comprising the nucleic acid molecule of claim 5.

8. A host cell comprising the expression vector of claim 7.

9. The host cell of claim 8 wherein the host cell is a B. subtilis.

10. A method of removing a target polypeptide from a sample comprising:
i) providing a sample comprising the target polypeptide; and
ii) adding the polypeptide of claim 1 to said sample for a sufficient period of time and at a suitable temperature to remove the target polypeptide.

11. The method of claim 10 further comprising increasing the temperature of the sample to inactivate the polypeptide.

12. The method of claim 11 wherein the temperature is increased to about 50° C.

13. The method of claim 10 wherein the target polypeptide is a polypeptide used in molecular biology techniques.

14. The method of claim 13 wherein the polypeptide used in molecular biology techniques is a heat resistant enzyme.

15. The method of claim 13 wherein the polypeptide used in molecular biology techniques is a nuclease, a DNA modifying enzyme, or a restriction enzyme.

16. The method of claim 10 wherein the sample is a preparation of plasmid DNA, a preparation of chromosomal DNA, a preparation of mitochondrial DNA, a preparation of RNA, a forensic sample, a clinical sample, or a diagnostic sample.

17. The method of claim 10 wherein the target polypeptide is a contaminant.

18. A composition comprising the polypeptide of claim 1 and a carrier.

19. The composition of claim 18 wherein the composition is a detergent composition.


SEQUENCE LISTING (22 sequences)

SEQ ID NO: 1 - DNA, 1244 bp, Artificial Sequence
  variant subtilisin Carlsberg sequence
acagcccaagctttctagagtccatataggataggagattgatgtatgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaacatcaccaccatcaccattaatgagatcaacagtttgggcagttgacggatccggggaattc

SEQ ID NO: 2 - protein, 379 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 3 - DNA, 1158 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaggcttataacaccgacggcaacggacacggcacacatgttgccggtacagtagctgcgcttggcaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcggctttgatcatgtcaaaacatccgaacctttcagcttcacaagtccgcaaccgtctctccagcacggcgacttatttgggaagctccttctactatgggaaaggtctgatcaatgtcgaagctgccgctccacatcaccaccatcaccattaa

SEQ ID NO: 4 - DNA, 1140 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaggcttataacaccgacggcaacggacacggcacacatgttgccggtacagtagctgcgcttggcaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcggctttgatcatgtcaaaacatccgaacctttcagcttcacaagtccgcaaccgtctctccagcacggcgacttatttgggaagctccttctactatgggaaaggtctgatcaatgtcgaagctgccgctccataa

SEQ ID NO: 5 - DNA, 1158 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaggcttataacaccgacggcaacggacacggcacacatgttgccggtacagtagctgcgcttgataatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcggctttgatcatgtcaaaacatccgaacctttcagcttcacaagtccgcaaccgtctctccagcacggcgacttatttgggaagctccttctactatgggaaaggtctgatcaatgtcgaagctgccgctccacatcaccaccatcaccattaa

SEQ ID NO: 6 - DNA, 1140 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaggcttataacaccgacggcaacggacacggcacacatgttgccggtacagtagctgcgcttgataatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcggctttgatcatgtcaaaacatccgaacctttcagcttcacaagtccgcaaccgtctctccagcacggcgacttatttgggaagctccttctactatgggaaaggtctgatcaatgtcgaagctgccgctccataa

SEQ ID NO: 7 - DNA, 1158 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaggcttataacaccgacggcaacggacacggcacacatgttgccggtacagtagctgcgcttgcaaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcggctttgatcatgtcaaaacatccgaacctttcagcttcacaagtccgcaaccgtctctccagcacggcgacttatttgggaagctccttctactatgggaaaggtctgatcaatgtcgaagctgccgctccacatcaccaccatcaccattaa

SEQ ID NO: 8 - DNA, 1140 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaggcttataacaccgacggcaacggacacggcacacatgttgccggtacagtagctgcgcttgcaaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcggctttgatcatgtcaaaacatccgaacctttcagcttcacaagtccgcaaccgtctctccagcacggcgacttatttgggaagctccttctactatgggaaaggtctgatcaatgtcgaagctgccgctccataa

SEQ ID NO: 9 - protein, 385 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALGNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALIMSKHPNLSASQVRNRLSSTATYLGSSFYYGKGLINVEAAAPHHHHHH

SEQ ID NO: 10 - protein, 379 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALGNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALIMSKHPNLSASQVRNRLSSTATYLGSSFYYGKGLINVEAAAP

SEQ ID NO: 11 - protein, 385 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALIMSKHPNLSASQVRNRLSSTATYLGSSFYYGKGLINVEAAAPHHHHHH

SEQ ID NO: 12 - protein, 379 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALIMSKHPNLSASQVRNRLSSTATYLGSSFYYGKGLINVEAAAP

SEQ ID NO: 13 - protein, 385 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALANTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALIMSKHPNLSASQVRNRLSSTATYLGSSFYYGKGLINVEAAAPHHHHHH

SEQ ID NO: 14 - protein, 379 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALANTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALIMSKHPNLSASQVRNRLSSTATYLGSSFYYGKGLINVEAAAP

SEQ ID NO: 15 - DNA, 1158 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaacaatgatccagatgtcgcttatgtggaagaggatcatgtggcccatgccttggcgcaaaccgttccttacggcattcctctcattaaagcggacaaagtgcaggcccaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttggctatacaacgggtgtattaggcgttgcgccaagcgtatccttgtacgcggttaaagtactgaattcaagcgggagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaagcacgaatacaattggctatcctgcgaaatacgattctgtcatcgctgttggtgcggtagactctaacagcaacagagcttcattttccagtgtgggagcagagcttgaagtcatggcccctggcgcaggcgtatacagcacttacccaacgaacacttatgcaacatttaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaacatcaccaccatcaccattaa

SEQ ID NO: 16 - DNA, 1140 bp, Artificial Sequence
  variant subtilisin Carlsberg sequence
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaacaatgatccagatgtcgcttatgtggaagaggatcatgtggcccatgccttggcgcaaaccgttccttacggcattcctctcattaaagcggacaaagtgcaggcccaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttggctatacaacgggtgtattaggcgttgcgccaagcgtatccttgtacgcggttaaagtactgaattcaagcgggagcggatcatacagcggcattgtaagcggaatcgagtgggcgacaacaaacggcatggatgttatcaatatgagccttgggggagcatcaggctcgacagcgatgaaacaggcagtcgacaatgcatatgcaaaaggggttgtcgttgtagctgcagcagggaacagcggatcttcaggaagcacgaatacaattggctatcctgcgaaatacgattctgtcatcgctgttggtgcggtagactctaacagcaacagagcttcattttccagtgtgggagcagagcttgaagtcatggcccctggcgcaggcgtatacagcacttacccaacgaacacttatgcaacatttaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaataa

SEQ ID NO: 17 - protein, 385 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVNNDPDVAYVEEDHVAHALAQTVPYGIPLIKADKVQAQGFKGANVKVAVLDTGIQASHPDLNVVGGASFVAGEAYNTDGNGHGTHVAGTVAALGYTTGVLGVAPSVSLYAVKVLNSSGSGSYSGIVSGIEWATTNGMDVINMSLGGASGSTAMKQAVDNAYAKGVVVVAAAGNSGSSGSTNTIGYPAKYDSVIAVGAVDSNSNRASFSSVGAELEVMAPGAGVYSTYPTNTYATFNGTSMASPHVAGAAALILSKHPNLSASQVRNRLSSTATYLGSSFYYGKGLINVEAAAQHHHHHH

SEQ ID NO: 18 - protein, 379 aa, Artificial Sequence
  variant subtilisin Carlsberg sequence
MMRKKSFWLGMLTAFMLVFTMAFSDSASAAQPAKNVEKDYIVGFKSGVKTASVKKDIIKESGGKVDKQFRIINAAKAKLDKEALKEVNNDPDVAYVEEDHVAHALAQTVPYGIPLIKADKVQAQGFKGANVKVAVLDTGIQASHPDLNVVGGASFVAGEAYNTDGNGHGTHVAGTVAALGYTTGVLGVAPSVSLYAVKVLNSSGSGSYSGIVSGIEWATTNGMDVINMSLGGASGSTAMKQAVDNAYAKGVVVVAAAGNSGSSGSTNTIGYPAKYDSVIAVGAVDSNSNRASFSSVGAELEVMAPGAGVYSTYPTNTYATFNGTSMASPHVAGAAALILSKHPNLSASQVRNRLSSTATYLGSSFYYGKGLINVEAAAQ

SEQ ID NO: 19 - DNA, 24 bp, Artificial Sequence
  Primer
tcagcccaagctttctagagtcca

SEQ ID NO: 20 - DNA, 21 bp, Artificial Sequence
  Primer
gaattccccggatccgtcaac

SEQ ID NO: 21 - DNA, 43 bp, Artificial Sequence
  Primer
atcaatctcctatcctatatggactctagaaagcttgggctga

SEQ ID NO: 22 - DNA, 41 bp, Artificial Sequence
  Primer
tgagatcaacagtttgggcagttgacggatccggggaattc